(19) United States
(12) Patent Application Publication
(10) Pub. No.: US 2021/0330504 A1
(43) Pub. Date: Oct. 28, 2021

(54) FACE SHIELD ASSEMBLY

(71) Applicant: ZVerse, Inc., Columbia, SC (US)

(72) Inventors: John Carrol Carrington, JR., Columbia, SC (US); Scott A. Henderson, Brooklyn, NY (US)

(21) Appl. No.: 17/238,588

(22) Filed: Apr. 23, 2021

Related U.S. Application Data
(60) Provisional application No. 63/030,736, filed on May 27, 2020.
(60) Provisional application No. 63/014,400, filed on Apr. 23, 2020.

(51) Int. Cl. A61F 9/04

(52) U.S. Cl. CPC A61F 9/045

(57) ABSTRACT

A face shield assembly comprises a holder adapted to be worn on the body of a wearer. The holder has a support portion and a separate attachment portion connected to the support portion. The support portion of the holder defines an opening through which the wearer's neck is received or, in another embodiment, on the head of the wearer. In addition, a shield is removably connected to the attachment portion of the holder, the shield being formed of a transparent material. The shield further has a curved configuration around a generally vertical axis as well as being narrower at one end than the other.

### PRIORITY CLAIM

This application is based upon and claims priority to U.S. provisional application Ser. No. 63/014,400 entitled “Food Service Industry Worker Face Shield Assembly,” filed Apr. 23, 2020, and U.S. provisional application Ser. No. 63/030,736 entitled “Food Service Industry Worker Face Shield Assembly,” filed May 27, 2020. Both of the aforementioned applications are incorporated herein by reference in their entirety for all purposes.

### FIELD OF THE INVENTION

The present invention relates generally to retail and food service industries. More particularly, this invention relates to a face shield that may be worn by a worker to inhibit transmission of microbes (e.g, bacteria and viruses) to and from the worker such as between the worker and a customer.

### BACKGROUND OF THE INVENTION

Workers in various industries that deal with the public have not typically worn any kind of protective masks when interacting with customers. For example, such workers in the food service industry might include seating hosts, wait staff, fast food cashiers, food preparers, and the like. In view of the global pandemic caused by the novel coronavirus, masks are now common. This is expected to continue at least for the foreseeable future.

Traditional medical face masks partially cover the face of the wearer. If such masks are worn by a worker interacting with a customer, the customer will not be able to determine whether the worker has a friendly or unfriendly demeanor. From the standpoint of a business owner, such masks thus create a psychological barrier to the welcoming and pleasant experience that the business owner seeks to provide.

Room exists in the art for an alternative face shield device that is particularly advantageous in a retail, hospitality, etc. situation.

### SUMMARY OF THE INVENTION

The present invention recognizes and addresses the foregoing considerations, and others, of prior art construction and methods.

One aspect of the present invention provides a face shield assembly. The assembly comprises a holder adapted to be worn on the body of a wearer, the holder having an attachment portion. A shield is removably connected to the attachment portion of the holder. The shield in this aspect is formed of a transparent material and is sized to extend from below the wearer's chin to a location above the nostrils of the wearer.

In some exemplary embodiments, the holder has a support portion separate from the attachment portion. For example, the attachment portion may be pivotally connected to the support portion such as by a hinge formed by engagement of the attachment portion with an axle of the support portion. Moreover, the support portion may be in the form of a loop defining an opening through which the wearer's neck is received. The support portion in such embodiments may be removably connected to the attachment portion for donning and doffing of the face shield assembly. In other embodiments, the support portion may have a pair of flexible extensions that curve toward one another at a location distal from the shield.

The shield of the face shield assembly may preferably be formed of a transparent polymeric material such as PET or polycarbonate. In some exemplary embodiments, the shield has a semi-cylindrical shape when attached to the holder. In addition, the shield may preferably be narrower at its bottom and wider at its top. For example, the shield may have a configuration that continuously diverges from its bottom to its top. In addition, the attachment portion may have a curved shape in conformance with a curved outer surface of the shield. For example, a curved shape of the shield may be maintained in some embodiments by the attachment portion. In some exemplary embodiments, the attachment portion may have at least one post received in an aperture in the shield to connect the shield to the attachment portion.

Another aspect of the present invention provides a face shield assembly comprising a holder adapted to be worn on the body of a wearer. According to this aspect, the holder has a support portion and a separate attachment portion connected to the support portion. The support portion of the holder defines an opening through which the wearer's neck is received. In addition, a shield is removably connected to the attachment portion of the holder, the shield being formed of a transparent material. The shield further has a curved configuration around a generally vertical axis as well as being narrower at its bottom and wider at its top.

A still further aspect of the present invention provides a face shield assembly comprising a holder adapted to be worn on the head of a wearer, the holder having an attachment portion. A shield is removably connected to the attachment portion of the holder, the shield being formed of a transparent material and sized to extend from above the wearer's eyes to a location at or below the chin of the wearer.

The accompanying drawings, which are incorporated in and constitute a part of this specification, illustrate one or more embodiments of the present invention.

### BRIEF DESCRIPTION OF THE DRAWINGS

A full and enabling disclosure of the present invention, including the best mode thereof directed to one of ordinary skill in the art, is set forth in the specification, which makes reference to the appended drawings, in which:

FIG. 1 shows a face shield assembly in accordance with an embodiment of the present invention being worn by a worker such as in the food service industry;

FIG. 2 is a perspective view of a face shield assembly in accordance with an embodiment of the present invention;

FIG. 3 is a left side elevational view of the face shield assembly of FIG. 2;

FIG. 4A is generally a rear elevational view of the face shield assembly of FIG. 2;

FIG. 4B is a front perspective view of the face shield assembly of FIG. 2 with the shield separated from the holder;

FIG. 5 is an enlarged fragmentary view showing attachment of the removable shield to the attachment portion of the holder;

FIG. 6 is an enlarged partial cross-sectional view showing a post of the holder extending through an aperture in the shield to secure the shield in position on the holder;

FIG. 7 is an enlarged fragmentary view showing a hinge on the holder that permits rotation of the attachment portion (and attached shield) relative to the support portion of the holder;

FIG. 8 diagrammatically illustrates rotation of the shield between active and idle positions;

FIG. 9 is an enlarged exploded view showing a preferred mechanism for allowing rotation of the shield relative to the holder;

FIG. 10 is a perspective view of the face shield assembly of FIG. 2 showing the support portion unclasped for donning or doffing;

FIG. 11 is a perspective view of a face shield assembly in accordance with another embodiment of the present invention;

FIG. 12 is generally a rear elevational view of the face shield assembly of FIG. 11;

FIG. 13 is an enlarged fragmentary view showing a hinge on the holder in accordance with the embodiment of FIG. 11;

FIG. 14 is an enlarged fragmentary view of the embodiment of FIG. 11 showing the support portion and holder separated;

FIG. 15 shows the embodiment of FIG. 11 equipped with an optional hanging hook;

FIG. 16 is an enlarged perspective view of the hanging hook;

FIG. 17 is a perspective view of a face shield assembly in accordance with another embodiment of the present invention being worn; and

FIG. 18 is a top perspective view of the face shield assembly of FIG. 17.

Repeat use of reference characters in the present specification and drawings is intended to represent same or analogous features or elements of the invention.

### DETAILED DESCRIPTION OF PREFERRED EMBODIMENTS

Reference will now be made in detail to presently preferred embodiments of the invention, one or more examples of which are illustrated in the accompanying drawings. Each example is provided by way of explanation of the invention, not limitation of the invention. In fact, it will be apparent to those skilled in the art that modifications and variations can be made in the present invention without departing from the scope or spirit thereof. For instance, features illustrated or described as part of one embodiment may be used on another embodiment to yield a still further embodiment. Thus, it is intended that the present invention covers such modifications and variations as come within the scope of the appended claims and their equivalents.

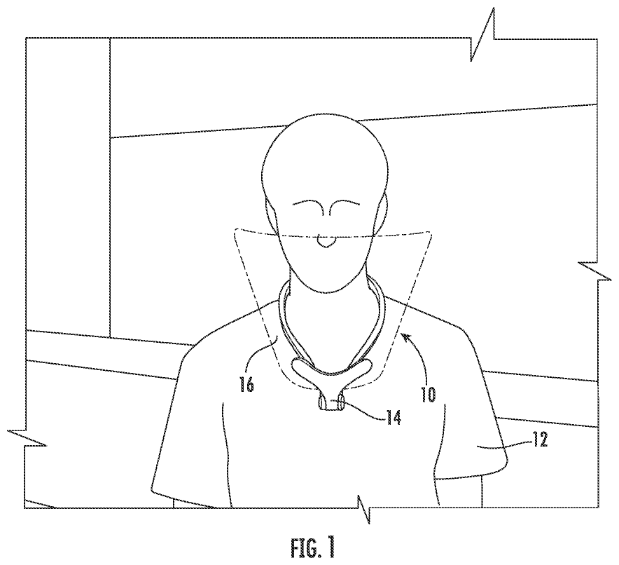

FIG. 1 illustrates a face shield assembly 10 in accordance with an embodiment of the present invention being worn by a worker 12. In this particular case, for example, the worker 10 may be a greeter or a cashier in a fast food restaurant. One skilled in the art will appreciate, however, that face shields of the present invention may be worn by a wide variety of people in various roles. For example, the face shield assembly 10 may be useful in various other retail or hospitality situations.

The face shield assembly 10 in this embodiment has a holder 14 which is worn on the body of worker 12 to support a face shield 16 that extends up to a location in front of the worker's mouth and nose (e.g., to a location at least above the wearer's nostrils). In many cases, it will be desirable for the face shield 16 to extend to a location above the wearer's eyes. Preferably, the shield 16 will be transparent to minimize its noticeability to customers and maximize the wearer's ability to see his or her surroundings. (The shields in the various embodiments herein are shown in dot-dashed lines to indicate transparency.) The presence of the shield 16 in this location inhibits transmission of microbes between the worker and customer, and also reassures the customer that the business is taking the appropriate precautions.

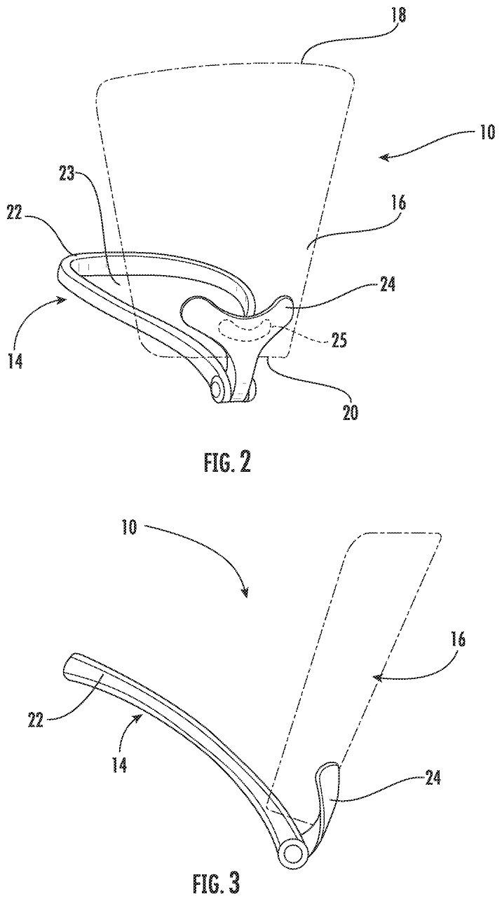

Referring now also to FIGS. 2-4B, additional details regarding face shield assembly 10 can be most easily explained. In this embodiment, shield 16 is curved (e.g., semi-cylindrical) in the vertical direction so that it generally “wraps around” the worker's face. As shown, shield 16 may be wider at its top 18 and narrower at its bottom 20. While any suitable configuration could be used, shield 16 continuously diverges from narrow to wide in this embodiment. In many embodiments, shield 16 may have a top to bottom length of at least eight inches, such as a top to bottom length falling in a range of ten to twelve inches. Shield 16 may be formed of any suitable polymeric or non-polymeric transparent material (e.g., glass). In this embodiment, for example, shield 16 may be formed of PET or polycarbonate, which are inexpensive and widely available materials (e.g., from recyclers of soda bottles).

Holder 14 may have any suitable configuration to be worn or anchored by the wearer's body so as to support shield 16 in the correct position. In this embodiment, for example, shield 16 has a support portion 22 in the form of a loop that defines a neck opening 23. In particular, opening 23 is sized to receive the worker's neck so that support portion 22 can rest on the worker's shoulders (as shown in FIG. 1). In this regard, support portion 22 will preferably have a contour that follows the human form from shoulder to chest (as can be most easily seen in FIG. 3). Shield 16 is attached to holder 14 at an attachment portion 24 that extends upward from support portion 22. Preferably, holder 14 may be formed of any suitable rigid or semi-rigid material(s), such as a resilient polymer by itself or a resilient polymer surrounding a harder core of rigid plastic or metal wire. In some exemplary embodiments, holder 14 may be formed entirely of polypropylene.

Attachment portion 24 is desirably made small (relative to the surface area of shield 16) so that it will preferably be entirely below the wearer's chin when worn. In addition, support portion 22 and attachment portion 24 are preferably configured so that at least part of attachment portion 24 will be located in front of the wearer's chin (i.e., it will be forward of the chin on a posterior to anterior plane bisecting the body). Advantageously, the area 25 on the front of attachment portion 24 may be used to display desired indicia, such as a name, logo, or other message (e.g., “hello!”) that the business wishes to convey. Notably, attachment portion 24 in this embodiment has a curved shape that forms or follows the contour of the outer surface (i.e., the surface away from the wearer) of shield 16. In some exemplary embodiments, attachment portion 24 may be formed of any suitable polymeric material, such as polypropylene.

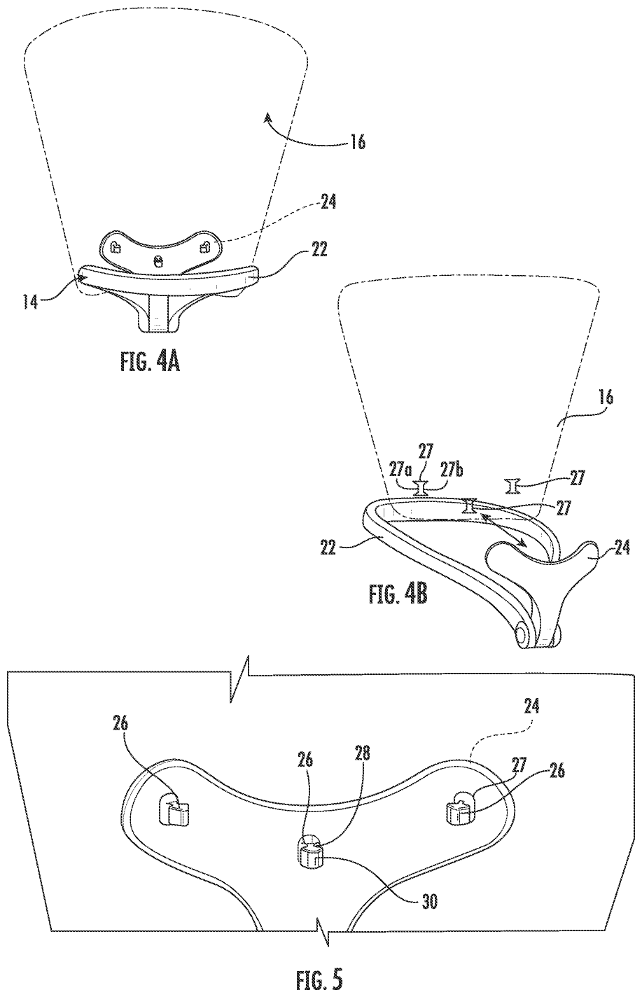

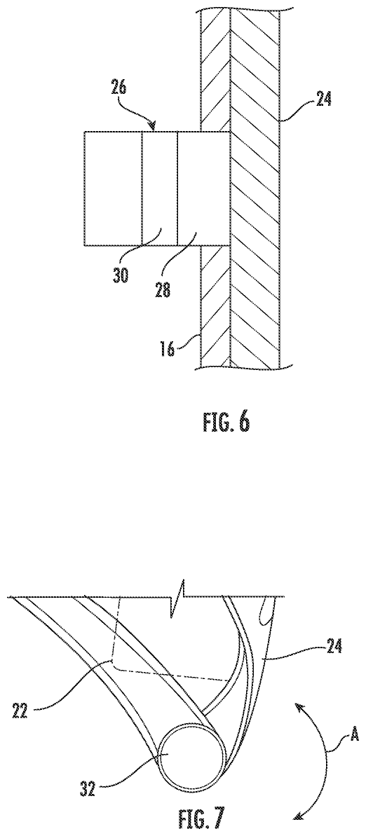

As shown in FIG. 4B, shield 16 is preferably removable from attachment portion 24 so that it can be more effectively cleaned or replaced. One skilled in the art will appreciate that any suitable means of removable attachment may be used, including fasteners, light adhesive, various snapping arrangements, etc. Referring now also to FIGS. 5 and 6, attachment portion 24 in this embodiment has a plurality of rearwardly-extending posts 26 that are received in corresponding apertures 27 in shield 16. The posts 26 may be formed having a proximal narrower portion 28 at which the shield 16 seats and a distal widened portion 30 that retains the shield in place. Preferably, the widened portion 30 may have forwardly-facing and rearwardly-facing sloped surfaces (e.g., forming a profile resembling an arrowhead) that facilitate attachment and removal of the shield 16. As shown in FIG. 4B, apertures 27 may be formed as “H-cuts” in which left and right flaps 27a and 27b deflect during insertion of the post 26.

Shield 16 may be formed from plastic sheet such that it has a flat shape prior to attachment to the attachment portion 24. As one skilled in the art will appreciate, shield 16 in such cases may be easily flexible such that it can be curved by the user as it is being attached. Due to the curve of attachment portion 24, shield 16 will maintain the desired curved configuration (i.e., wrapping around the wearer's face) once attached.

In some exemplary embodiments, attachment portion 24 may be formed as a separate piece that is connected to support portion 22. For example, as shown in FIG. 7, attachment portion 24 may be pivotally connected to support portion 22 via a suitable hinge 32. Preferably, the hinge 32 will be configured to resist rotation in the absence of intentional action. For example, hinge 32 may provide sufficient friction so that shield 16 will not rotate unless the wearer intentionally rotates attachment portion 24, as shown by arrow A, with respect to the support portion 22. The wearer can thus adjust the position of shield 16 as necessary or desired.

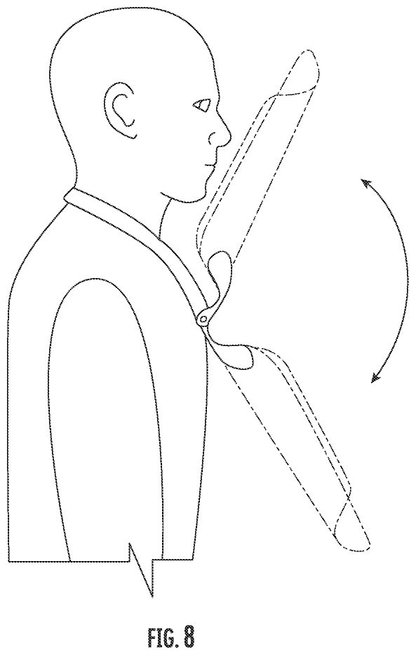

In presently preferred embodiments, shield 16 can be angled completely away from the face to allow the wearer to do things like eat or take a phone call without having to remove face shield assembly 10 completely. For example, as shown in FIG. 8, shield 16 may be moved all the way down into a non-use resting (“idle”) position when desired. Subsequently, the shield is pivoted back to the upper active position.

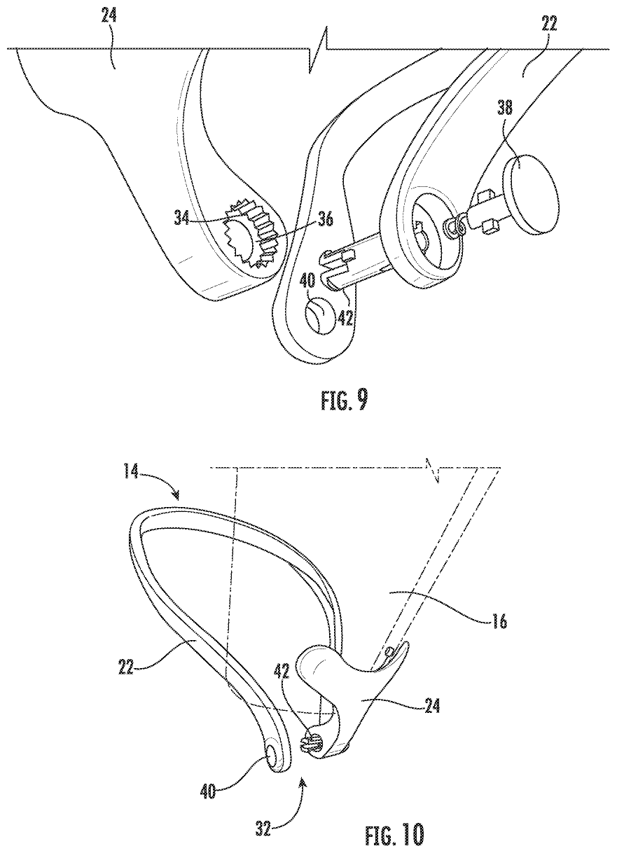

Toward this end, hinge 32 may have a series of detents at different angular positions to facilitate adjustment. In another embodiment, as shown in FIG. 9, there are a series of geared teeth 34 on the inside left of the attachment portion 24. A clutch ring 36 in this embodiment engages the teeth inside the attachment portion 24. The clutch ring 36 gets pushed free by a spring-loaded button 38, allowing attachment portion 24 to freely rotate. When the spring-loaded button 38 is released, the clutch-ring reengages the teeth inside attachment portion 24, thus locking attachment portion 24 into the desired angle.

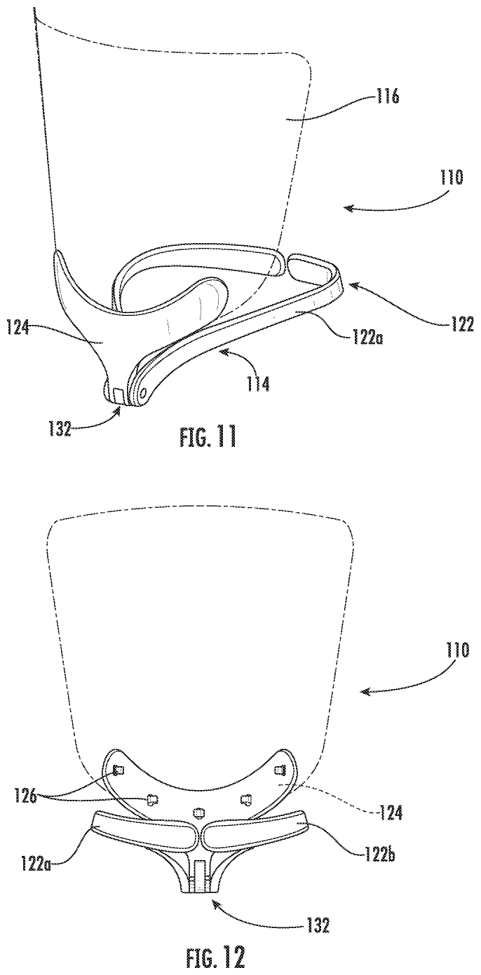

FIG. 10 illustrates support portion 22 in an open (not clasped) position for putting on and taking off of the face shield assembly 10. To put on (don) face shield assembly 10, the loop form of support portion 22 is wrapped around the wearer's neck and clasped. Face shield assembly 10 is taken off (doffed) using the opposite procedure. Preferably, clasping will occur in the front of the wearer's body so that the wearer can easily do it without the assistance of another person (which might be necessary if clasping occurred at the wearer's back).

In this embodiment, clasping and unclasping occurs at hinge 32. Specifically, a hole 40 defined in the end of support portion 22 cooperates with flex tabs 42 integrally formed on attachment portion 24. For clasping, the wearer simply moves support portion 22 such that the flex tabs 42 are forced into hole 40. (Preferably, the clasping is light such that the user can engage the flex tabs 42 without squeezing the flex tabs themselves.) The flex tabs may chamfer around the outer side of hole 40.

FIGS. 11 and 12 illustrate a face shield assembly 110 in accordance with another embodiment of the present invention. Assembly 110 is similar in many respects to face shield assembly 10 discussed above and may be made from similar materials. As such, similar elements will be designated with a reference number augmented by one hundred. Thus, assembly 110 includes a holder 114 to which a shield 116 is attached. In this embodiment, holder 114 has a support portion 122 with an overall contour similar to support portion 22 but having a pair of flexible extensions 122a and 122b that extend around the wearer's neck to meet (or nearly meet) at the back of the neck. Together, extensions 122a and 122b thus define an opening in which the wearer's neck is located when face shield assembly 110 is worn. Because extensions 122a and 122b are flexible, assembly 110 can be donned and doffed by simply spreading extensions 122a and 122b apart as they are positioned on sides of the wearer's neck. When the spreading force is released, extensions 122a and 122b will tend to assume an at rest position (as shown) with the ends of extensions 122a and 122b behind the neck. One advantage of this arrangement is that it allows easy “tear away” of assembly 110 if necessary or desired to remove it quickly.

Shield 116 is attached to holder 114 at an attachment portion 124 that extends upward from support portion 122. Attachment portion 124 in this embodiment has a plurality of rearwardly-extending posts 126 that are received in corresponding apertures 127 in shield 116. In this case, there are five such posts and apertures but other numbers such as seven posts and apertures could be used.

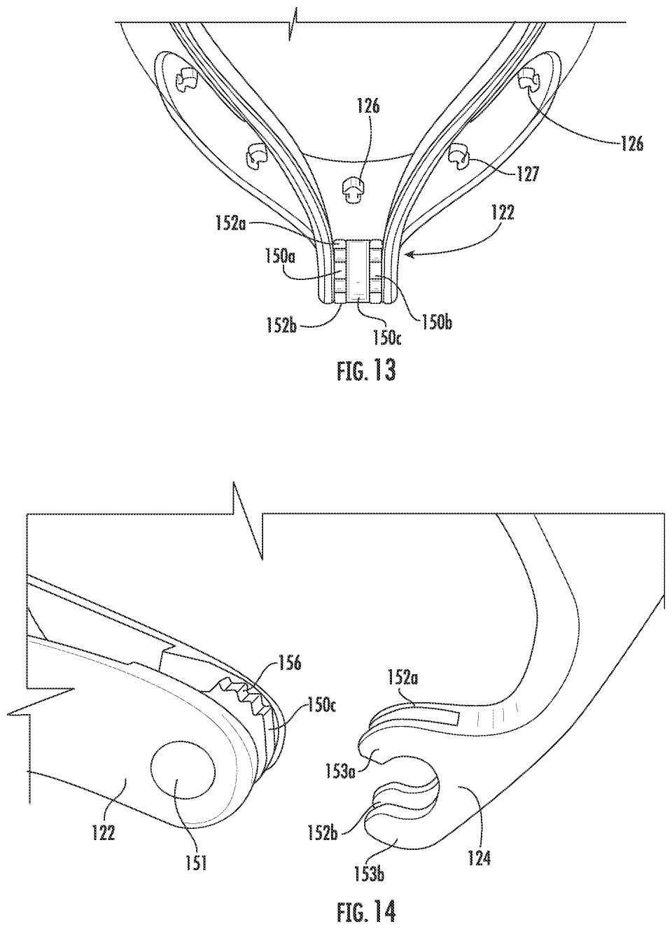

Attachment portion 124 is pivotally connected to support portion 122 via a hinge 132 for minor adjustment of shield position as well as movement of the shield between active and idle positions. Referring now particularly to FIGS. 13 and 14, the hinge 132 is formed in this example by the configuration of support portion 122 and attachment portion 124. In particular, support portion 122 defines an integral axle which extends when worn transverse to the wearer's chest below the inner ends of the clavicles. Preferably, the axle may have a pair of reduced diameter portions 150a and 150b on either side of an enlarged diameter disc 150c. The axle may be hollow such that it defines a passage from one side to the other with open ends, such as open end 151 (FIG. 14).

The reduced diameter portions 150a and 150b are engaged by flexible arms 152a-b and 153a-b that are an integral part of attachment portion 124. Arms 152a-b and 153a-b may preferably form C-shaped structures such that they can be press fit onto the respective reduced diameter portions 150a and 150b. As a result, assembly 110 is easy to put together by simply connecting attachment portion 124 to support portion 122. (In addition to the shield 116, assembly 110 thus has only two parts.) As with the previous embodiment, hinge 132 preferably resists rotation in the absence of intentional action. For example, a detent arrangement may be used to allow adjustment of the angle of the shield. The detent arrangement may, for example, comprise bumps or ridges 156 formed into the outer annular surface of disc 150c that interact with a similar feature on the adjacent inner surface of support portion 122.

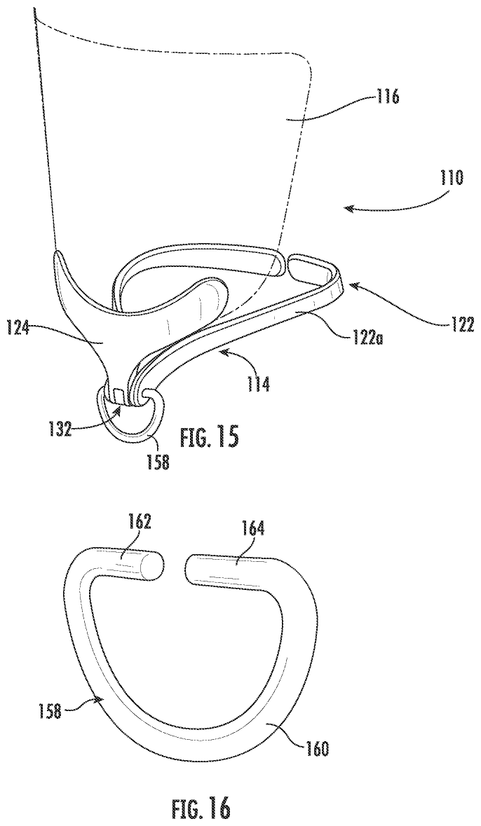

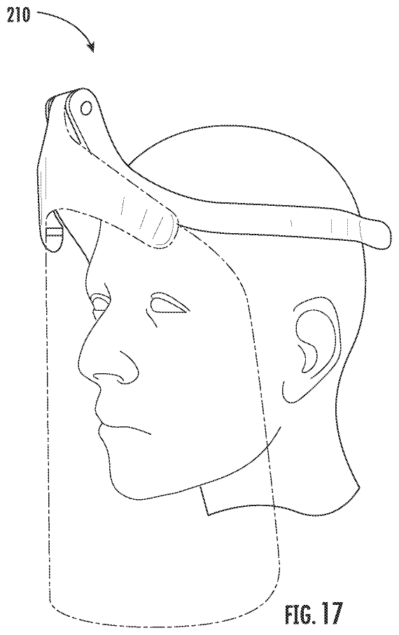

Referring now to FIGS. 15 and 16, assembly 110 (and other embodiments in accordance with the present invention) may be equipped with a hanging hook for stowing the face shield assembly when not in use. In this case, for example, a removable hanging hook 158 has a U-shaped portion 160 with opposed ends 162 and 164. Hook 158 is flexible, allowing ends 162 and 164 to be gently separated for insertion into or removal from respective sides of the hinge axle.

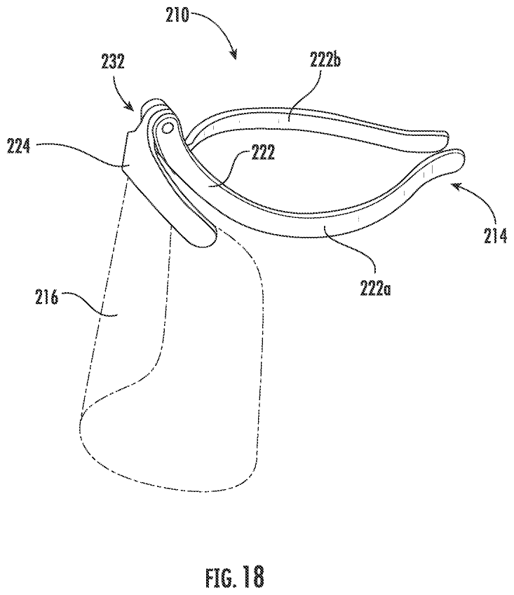

FIGS. 17 and 18 illustrate a face shield assembly 210 in accordance with a further embodiment of the present invention. Assembly 210 is similar in many respects to the face shield assembly 110 discussed above and may be made from similar materials. As such, similar elements will be designated with a reference number augmented by one hundred (i.e., two hundred more than face shield assembly 10). In this case, however, face shield assembly 210 is intended to be worn on the head of a wearer such that the shield 216 depends from a location above the wearer's eyes. Preferably, shield 216 extends to a location at least to or below the chin of the wearer.

Referring particularly to FIG. 18, assembly 210 includes a holder 214 to which the shield 216 is attached. Holder 214 has a support portion 222 having a pair of flexible extensions 222a and 222b that extend around to the back of the wearer's head. The assembly 210 may be donned, for example, by pushing onto the wearer's head from above, causing extensions 222a and 222b to spread. Because extensions 222a and 222b tend to retain their at rest position, they are lightly urged against the sides of the wearer's head when worn by material memory. As a result, assembly 210 tends to stay in place. At the same time, assembly 210 is easy to remove when desired.

Shield 216 is attached to holder 214 at an attachment portion 224 that extends downward from support portion 222. Preferably, shield 216 is connected to the attachment portion 224 in a manner similar to the previous embodiments. In addition, attachment portion 224 is preferably pivotally connected to support portion 222 via a hinge 232 that is similar to hinge 132. In this case, however, the hinge is constructed so that attachment portion 224 can rotate to a position that is substantially vertically downward, as shown.

It can thus be seen that the present invention provides a novel face shield assembly for use by individuals wishing to inhibit transmission of microbes. While one or more preferred embodiments of the invention have been described above, it should be understood that any and all equivalent realizations of the present invention are included within the scope and spirit thereof. The embodiments depicted are presented by way of example only and are not intended as limitations upon the present invention. Thus, it should be understood by those of ordinary skill in this art that the present invention is not limited to these embodiments since modifications can be made. Therefore, it is contemplated that any and all such embodiments are included in the present invention as may fall within the scope and spirit thereof.

What is claimed is:

1. A face shield assembly, comprising:
a holder adapted to be worn on the body of a wearer, said holder having an attachment portion; and
a shield removably connected to the attachment portion of said holder, said shield being formed of a transparent material and is sized to extend from below the wearer's chin to a location above the nostrils of the wearer.

2. A face shield assembly as set forth in claim 1, wherein the holder has a support portion separate from the attachment portion.

3. A face shield assembly as set forth in claim 2, wherein the attachment portion is pivotally connected to the support portion.

4. A face shield assembly as set forth in claim 3, wherein the support portion is removably connected to the attachment portion for donning and doffing the face shield assembly.

5. A face shield assembly as set forth in claim 2, wherein the support portion is in the form of a loop defining an opening through which the wearer's neck is received.

6. A face shield assembly as set forth in claim 2, wherein the support portion has a pair of flexible extensions that curve toward one another at a location distal from the shield.

7. A face shield assembly as set forth in claim 1, wherein the shield is formed of a transparent polymeric material.

8. A face shield assembly as set forth in claim 7, wherein the polymeric material comprises PET.

9. A face shield assembly as set forth in claim 1, wherein the shield has a semi-cylindrical shape when attached to the holder.

10. A face shield assembly as set forth in claim 9, wherein the shield is narrower at its bottom and wider at its top.

11. A face shield assembly as set forth in claim 9, wherein the attachment portion has a curved shape in conformance with a curved outer surface of the shield.

12. A face shield assembly as set forth in claim 11, wherein the curved outer surface of the shield is maintained by the attachment portion.

13. A face shield assembly as set forth in claim 1, wherein the shield has a configuration that continuously diverges from its bottom to its top.

14. A face shield as set forth in claim 1, wherein the attachment portion has at least one post received in an aperture in the shield to connect the shield to the attachment portion.

15. A face shield assembly, comprising:
a holder adapted to be worn on the body of a wearer, said holder having a support portion and a separate attachment portion connected to the support portion;
the support portion of the holder defining an opening through which the wearer's neck is received;
a shield removably connected to the attachment portion of said holder, said shield being formed of a transparent material; and
the shield having a curved configuration around a generally vertical axis, the face shield further being narrower at its bottom and wider at its top.

16. A face shield assembly as set forth in claim 15, wherein the attachment portion is pivotally connected to the support portion.

17. A face shield assembly as set forth in claim 16, wherein the attachment portion is pivotally connected to the support portion by a hinge formed by engagement of the attachment portion with an axle of the support portion.

18. A face shield assembly as set forth in claim 16, wherein the support portion is removably connected to the attachment portion for donning and doffing the face shield assembly.

19. A face shield assembly as set forth in claim 15, wherein the support portion has a pair of flexible extensions that curve toward one another at a location distal from the shield.

20. A face shield assembly, comprising:
a holder adapted to be worn on the head of a wearer, said holder having an attachment portion; and
a shield removably connected to the attachment portion of said holder, said shield being formed of a transparent material and is sized to extend from above the wearer's eyes to a location below the chin of the wearer.
